(19) United States
(12) Patent Application Publication
(10) Pub. No.: US 2021/0030787 A1
(43) Pub. Date: Feb. 4, 2021

(54) METHODS FOR PROTECTING HAIR FOLLICLES

(71) Applicant: Applied Biology, Inc., Irvine, CA (US)

(72) Inventors: Ofer A. Goren, Irvine, CA (US); John McCoy, Irvine, CA (US)

(21) Appl. No.: 16/947,423

(22) Filed: Jul. 31, 2020

Related U.S. Application Data
(60) Provisional application No. 62/880,940, filed on Jul. 31, 2019.

(51) Int. Cl. A61K 33/04; A61K 9/00; A61K 35/741; A61K 35/74; A61K 35/76; A61P 17/14; A61B 5/00; A61M 35/00; A61K 39/395

(52) U.S. Cl. CPC A61K 33/04; A61K 9/0014; A61K 35/741; A61K 35/74; A61K 39/39583; A61P 17/14; A61B 5/446; A61B 5/4848; A61M 35/003; A61K 35/76

(57) ABSTRACT

Disclosed herein are compositions including a topical agent that may be used to reduce and/or inhibit the effects of chemotherapy and/or radiotherapy, which can include reducing and/or inhibiting the effects of hair loss and/or hair shedding due to chemotherapy and/or radiotherapy. The topical agent can be hydrogen sulfide, a hydrogen sulfide donor, or a substrate for a hydrogen sulfide producing enzyme such as sulfanegen. Some embodiments of the composition can include the topical agent combined with an agent to promote vasoconstriction or the topical agent combined with an agent to modulate deposition, mode of action, and/or metabolism of a chemotherapeutic agent and/or a reactive oxygen species agent. Embodiments can be used for improving the outcome of a hair transplant surgery by optimizing a holding solution to stabilize hair follicle grafts. In yet another embodiment, a system for realizing hydrogen sulfide slowly into a holding solution is described.

### CROSS REFERENCE

This application claims the benefit of U.S. Provisional Application No. 62/880,940, filed on Jul. 31, 2019, which is incorporated by reference in its entirety.

### TECHNICAL FIELD

The present invention relates to methods of treating or preventing forms of alopecia by administering topical applications of compositions that can include a hydrogen sulfide donor. Some methods can include use of compositions to reduce or retard cellular metabolism and/or inhibit perfusion, which may facilitate treatment and/or prophylaxis of chemotherapy induced alopecia. Additionally, the present invention relates to methods for preserving hair follicles excised during hair replacement surgery.

### BACKGROUND

Chemotherapy induced alopecia (“CIA”) can be a frequent adverse event present in patients undergoing oncological treatment, with an estimated incidence of 65% (see, Trueb 2009). CIA can have serious consequences to cancer patients' survivability. For example, 8% of women reject chemotherapy due to the potential risk of developing CIA. In addition, the psychological burden of CIA can lower self-esteem, body image, and quality of life (see, McGarvey 2001). A prophylactic treatment for CIA may be of significant clinical benefit to patients undergoing oncological treatment.

Effective chemotherapeutic agents can differentially affect rapidly dividing cells, e.g., cancer cells. Unfortunately, other rapidly dividing but noncancerous cells, such as, stem cells in the hair follicle niche, can also be affected. As a result, a significant number of patients undergoing chemotherapy may develop CIA. Some strategies for inhibiting CIA have been previously explored.

In 2017, the U.S. Food and Drug Administration (“FDA”) approved a scalp cooling system for reducing hair loss in breast cancer patients undergoing chemotherapy. Scalp cooling has been proven to effectively reduce the incidence of CIA. In a study of scalp cooling (see, Nangia et al. 2017), hair preservation was observed in approximately 50% of women that used the scalp cooling device; none of the women in the control group preserved hair. While scalp cooling as a prophylactic treatment for CIA is effective in some patients, it is time consuming, often uncomfortable, and expensive. Additionally, the procedure is not widely available.

Scalp cooling during chemotherapy sessions, has two proposed mechanisms for reducing CIA. First, scalp cooling induces cutaneous vasoconstriction resulting in reduced uptake of chemotherapeutic agents by hair follicles (see, FDA press release). Additionally, it has been proposed that the metabolism of chemotherapeutic agents or other important intermediates is reduced in hair follicle cells at lowered temperatures. As such, topical agents that reduce blood perfusion and/or slow cellular metabolism could produce results similar or superior to scalp cooling.

Other means of achieving the benefits of scalp cooling have been explored. Soref and Fahl (2015), demonstrated that topically applied vasoconstrictors reduced the reach of chemotherapeutic agents into the hair follicle niche and subsequently reduced the likelihood of developing CIA in rodents. Additionally, U.S. Patent Publication No. 20160136113 describes the use of specific vasoconstrictors to provide protection against the adverse effects, e.g., alopecia, mucositis or dermatitis, induced by chemotherapy or radiotherapy.

Additionally, other modes of modulating the deposition or metabolism of chemotherapy agents locally have been proposed. These agents can act on a specific chemotherapeutic agent or class of chemotherapeutic agents and could be applied locally, e.g., to the scalp, to stop chemotherapeutic agents from acting on the area of application. Similarly, other compounds have been proposed that inhibit CIA by different mechanisms. Many of these agents arrest cell growth in stages that are not vulnerable to chemotherapy. A few examples of such agents are provided in Table 1.

TABLE 1
Examples of Agents That Arrest Cell Growth in Stages

| CIA Reducing Compound | Mode of Action | Chemotherapy | Reference |
|---|---|---|---|
| MAD11 monoclonal | Inhibition (direct | doxorubicin and other | Balsari et al., |
| antibody (MAb) | binding) of | anthracyclines | 1994 |
| | chemotherapeutic agent | | |
| Cyclosporine A | Inhibition of Go to | cyclophosphamide, cytosine | Hussein et al., |
| | G1 | arabinoside and etoposide | 1995 |
| N-acetyl cysteine | Antioxidant | cyclophosphamide | Wang et al., |
| | | | 2006 |
| alpha-tocopherol, | Antioxidant | doxorubicin | Batchelor, |
| beta-tocopherol, | | | 2001 |
| gamma-tocopherol, | | | |
| delta-tocopherol | | | |
| Calcitriol | Inhibition of Go to | cyclophosphamide, etoposide, | Jimenez and |
| | G1 | cyclophosphamide, | Yunis, 1996 |
| | | doxorubicin, and paclitaxel | |
| CDK2 inhibitor | Inhibition of G1 to | etoposide, 5-fluorouracil, | Davis et al., |
| | S | taxol, cisplatin, and | 2001 |
| | | doxorubicin | |
| 2,2′-methylenebis | Inhibition of | etoposide | Tsuda et al., |
| | Capsase-3 | | 2001 |
| Antioxidants | Reduction of ROS | Radiation Therapy | |
| Antioxidants | Reduction of ROS | anthracyclines (e.g., | Conklin, 2004 |
| | | doxorubicin, epirubicin, and | |
| | | daunorubicin), alkylating | |
| | | agents (e.g., | |
| | | cyclophosphamide), platinum | |
| | | coordination complexes (e.g., | |
| | | cisplatin, carboplatin, and | |
| | | oxaliplatin), and | |
| | | epipodophyllotoxins (e.g., | |
| | | etoposide) | |
| Dexamethasone | Up regulation of | docetaxel, paclitaxel | De Weger et al., |
| | CYP3A | | 2014 |
| Praeruptorin C | Up regulation of | taxanes | Huang et al., |
| | CYP3A | | 2013 |
| Rifampin, | Up regulation of | taxanes | |
| Carbamezapine, | CYP3A | | |
| Phenytoin, | | | |
| Phenobarbital, | | | |
| Corticosteroids, | | | |
| St. John's Wart | | | |
| Calcitriol-analogs | | cyclophosphamide | Schilli et al., |
| | | | 1998 |

Hair transplantation surgery is a common remedy for patients that suffer from hair loss (alopecia). Current hair transplantation techniques involve extracting intact hair follicles from a donor site, dissecting single follicular units from the extracted tissue, and transplanting them to balding scalp. The success of current hair transplantation techniques, e.g., strip method extraction and follicular unit extraction (FUE), can be limited by the follicular survival rate after implantation. Commonly, follicular units are harvested from the donor site in the anagen phase of the hair cycle. Transplanted follicles can experience shock (termed “shock hair loss”), which can lead to anagen hairs being pre-maturely converted to the telogen phase of the hair cycle after transplantation. The impact of shock hair loss on patients is that most of the transplanted hairs will be converted to the telogen phase and fall out soon after surgery. Although the follicular unit will remain intact and hair will eventually grow at the transplantation site, the results of hair surgery will not be appreciated until many months after the procedure. Reducing shock hair loss would be of great benefit to patients undergoing hair transplantation surgery because patients would have full-length hair soon after recovering from surgery.

During hair transplantation surgery, hair-bearing skin is excised from the donor region either as strips, punch biopsy, or with specialized tools that can extract single follicular units. Suitable hair is commonly harvested from the occipital region of the scalp. Regardless of the method used, the excised grafts are placed in a chilled holding solution. Strip and punch biopsied hair is later removed from the holding solution and dissected into single follicular units and placed back into the holding solution. Excised follicular units have been reported to be viable in saline solutions for times up to 2 hours, however, keeping grafts in the holding solution for longer times impacts the success rate of the transplant surgery. A more expansive explanation of follicular viability degradation in holding solutions can be found in Avram, et. al (2017), which is incorporated here in its entirety.

Similar to holding time, the composition of the holding solution may be important to follicle survival rate and shock hair loss. It has been reported in the literature that different solutions affect graft viability, for example, Ringer's lactate solution has been found to be superior to saline. Additionally, a more complex medium containing various salts and glucose have been reported to be superior to Ringer's lactate solution in an in vitro test to determine viability using trypan blue. A complete description of these differences can be found in Gho, et. al (2014), which is incorporated here in its entirety. Additionally, specialized media have been designed to store and hold tissue and organs and lower temperatures. See for example, U.S. Pat. No. 6,045,990A, for a description of one such specialized media, which is incorporated here in its entirety. Transplantation survival rates as high as 97% have been reported using specialized media, however, hair shock loss remains a common problem.

Hydrogen sulfide (H₂S) has recently emerged as a member of the gasotransmitter family of signaling molecules along with nitric oxide and carbon monoxide. It has been shown to be a neurotransmitter and additionally to play a major role in many different physiological regulatory systems. In the skin, it has been suggested that H₂S regulates important functions, i.e., changes in its concentration are present in many skin pathologies.

Hydrogen sulfide is produced in mammals by the enzymatic metabolism of L-cysteine by cystathionine β-synthase (CBS) and cystathionine γ-lyase (CSE). CBS and CSE are pyridoxal 5′-phosphate dependent enzymes. CBS uses L-cysteine and homocysteine to produce H₂S. CSE catalyzes a cystathionine cleavage reaction to provide L-cysteine, ammonia, and α-ketobutyrate. Additionally, the enzymatic pathway containing cysteine aminotransferase and 3 -mercaptopyruvate sulfurtransferase (3MST) produce H₂S. Cysteine aminotransferase catalysis the production of 3-mercaptopyruvate from L-cysteine and α-ketoglutarate in the presence of reductants; 3MST acts on 3-mercaptopyruvate to produce H₂S. Additionally, the enzymes D-amino-oxidase and 3MST have been shown produce H₂S from D-cysteine. H₂S can also be generated by reduction of elemental sulfur by GSH involving NADH or NADPH as electron donors through glycolysis. Thiosulfate anions (S₂O₃²⁻) can also be reduced with pyruvate as a hydrogen donor to form H₂S.

Hydrogen sulfide has been proposed to have potential therapeutic effects on harvested hearts via diverse mechanisms. Its protective effects are reported to be primarily related to mitigating the inflammatory response and oxidative stress. Numerous studies have characterized hydrogen sulfide as an anti-inflammatory factor, and it has been demonstrated that hydrogen sulfide may protect against inflammation and apoptosis by inhibiting the TLR4/NF-κB pathway and NLRP3 activation in cardiomyocytes. In skin, topically applied hydrogen sulfate has been shown to produce varied effects on many dermal pathologies, e.g., inflammation, pruritus, psoriasis, melanoma, and wound healing (see, Coavoy-Sanchez, 2019). Additionally, hydrogen sulfide has been demonstrated to slow metabolism and render tissue in a state similar to that of cryopreservation.

### SUMMARY

The present invention concerns methods of treatment and/or prophylaxis of forms of alopecia by topical administration of a composition including a topical agent. In certain embodiments the topical agent reduces and or inhibits the effects of cancer treatment (e.g., chemotherapy, radiotherapy, etc.), which can include reducing and/or inhibiting the effects of hair loss and/or hair shedding due to chemotherapy and/or radiotherapy. The topical agent can be hydrogen sulfide, a hydrogen sulfide donor, or a substrate for a hydrogen sulfide producing enzyme molecule, e.g., dialyl trisulfide (DATS). Some embodiments of the composition can include a topical active agent with a modulating agent, the modulating agent being configured to modulate deposition, mode of action, and/or metabolism of a chemotherapeutic agent and/or a reactive oxygen species (“ROS”) agent. Some embodiments of the composition can be configured to reduce metabolism in rapidly dividing cells.

Embodiments can include an applicator kit. The applicator kit may include a spray device and/or a hair care product, such as a shampoo, conditioner, oil, etc. The application kit may include a container containing an embodiment of the composition and an applicator configured to test a region of the scalp of a person for perfusion.

In at least one embodiment, a method of treatment and/or prophylaxis of forms of alopecia can include applying a composition topically to a scalp of a person before, during, and/or after undergoing chemotherapy and/or radiation therapy. The composition may include a therapeutically effective amount of hydrogen sulfide, a hydrogen sulfide donor, or a substrate for a hydrogen sulfide producing enzyme. Some embodiments can include applying the composition to the person so as to reduce metabolism in local tissue. Some embodiments can include applying the composition to the person so that amounts of chemotherapeutic agents and/or reactive oxygen species agents delivered to a hair follicle is limited. Some embodiments can include applying the composition to maintain a therapeutically effective amount for a prolonged period of time. The prolonged period of time can be within a range from 1 hour to 48 hours. In some embodiments, the composition can further include a penetration enhancer. In some embodiments, the form of alopecia is chemotherapy induced alopecia and the chemotherapy is Taxane and/or Anthracycline-based chemotherapy. In some embodiments, the chemotherapy is used to treat stage I and/or stage II breast cancer patients.

In at least one embodiment, a method of treatment and/or prophylaxis of forms of alopecia can include applying a composition to the eyebrows of a person before, during, and/or after undergoing chemotherapy and/or radiation therapy, the composition comprising a therapeutically effective amount of hydrogen sulfide, a hydrogen sulfide donor, or a substrate for a hydrogen sulfide producing enzyme and a modulating agent, the modulating agent configured to up-regulate CYP3A, CSE, CBS, 3MST, or other hydrogen sulfide producing enzymes. In some embodiments, a method of treatment can include applying a composition to the fingernails of a person before, during, and/or after undergoing chemotherapy and/or radiation therapy. In other embodiments, the chemotherapy is Taxane and/or Anthracycline-based chemotherapy. In some embodiments, the chemotherapy is used to treat stage I and/or stage II breast cancer patients.

In at least one embodiment, a method of treatment and/or prophylaxis of forms of alopecia can include applying a composition to a scalp of a person before, during, and/or after undergoing chemotherapy and/or radiation therapy, the composition comprising a therapeutically effective amount of hydrogen sulfide, a hydrogen sulfide donor, or a substrate for a hydrogen sulfide producing enzyme and a modulating agent, wherein the modulating agent is configured to modulate deposition, mode of action, and/or metabolism of a chemotherapeutic agent and/or a reactive oxygen species agent. In some embodiments, the form of alopecia is chemotherapy induced alopecia and the chemotherapy is Taxane and/or Anthracycline-based chemotherapy. In some embodiments, the chemotherapy is used to treat stage I and/or stage II breast cancer patients.

In at least one embodiment, a method of treatment and/or prophylaxis of forms of alopecia can include applying a composition to a scalp of a person before, during, and/or after undergoing chemotherapy and/or radiation therapy, the composition comprising a therapeutically effective amount of a topical agent, wherein the topical agent is configured to up-regulate CYP3A, CSE, CBS, 3MST, or other hydrogen sulfide producing enzymes. In some embodiments, the form of alopecia is chemotherapy induced alopecia and the chemotherapy is Taxane and/or Anthracycline-based chemotherapy. In some embodiments, the chemotherapy is used to treat stage I and/or stage II breast cancer patients.

In at least one embodiment, a method of treatment and/or prophylaxis of forms of alopecia can include applying a composition to a scalp of a person before, during, and/or after undergoing chemotherapy and/or radiation therapy, the composition comprising a therapeutically effective amount of hydrogen sulfide, a hydrogen sulfide donor, or a substrate for a hydrogen sulfide producing enzyme and a modulating agent, the modulating agent configured to up-regulate CYP3A, CSE, CBS, 3MST, or other hydrogen sulfide producing enzymes. In some embodiments, the form of alopecia is chemotherapy induced alopecia and the chemotherapy is Taxane and/or Anthracycline-based chemotherapy. In some embodiments, the chemotherapy is used to treat stage I and/or stage II breast cancer patients.

In at least one embodiment, a kit for treatment and/or prophylaxis of chemotherapy induced alopecia can include a container containing a hydrogen sulfide donor or a substrate for a hydrogen sulfide producing enzyme. The kit may further include at least one of: an applicator configured to test a region of the scalp of a person for perfusion; and a disposable tip configured for use with a perfusion testing device.

In at least one embodiment, a kit for treatment and/or prophylaxis of forms of alopecia can include a container containing hydrogen sulfide, a hydrogen sulfide donor, or a substrate for a hydrogen sulfide producing enzyme and a modulating agent, wherein the modulating agent is configured to modulate deposition, mode of action, and/or metabolism of a chemotherapeutic agent and/or a reactive oxygen species agent. The kit may further include at least one of: an applicator configured to test a region of the scalp of a person for perfusion; and a disposable tip configured for use with a perfusion testing device.

In at least one embodiment, a kit for treatment and/or prophylaxis of forms of alopecia can include a container containing a topical agent. The topical agent can be configured to up-regulate CYP3A, CSE, CBS, 3MST, or other hydrogen sulfide producing enzymes. The kit may further include at least one of: an applicator configured to test a region of the scalp of a person for perfusion; and a disposable tip configured for use with a perfusion testing device.

In at least one embodiment, a kit for treatment and/or prophylaxis of forms of alopecia can include a container containing hydrogen sulfide, a hydrogen sulfide donor, or a substrate for a hydrogen sulfide producing enzyme and a modulating agent. The modulating agent can be configured to up-regulate CYP3A, CSE, CBS, 3MST, or other hydrogen sulfide producing enzymes. The kit may further include at least one of: an applicator configured to test a region of the scalp of a person for perfusion; and a disposable tip configured for use with a perfusion testing device.

In at least one embodiment, a hair care product for treatment and/or prophylaxis of forms of alopecia can include a pump spray container containing hydrogen sulfide, a hydrogen sulfide donor, or a substrate for a hydrogen sulfide producing enzyme.

In at least one embodiment, a hair care product for treatment and/or prophylaxis of forms of alopecia can include a pump spray container containing hydrogen sulfide, a hydrogen sulfide donor, or a substrate for a hydrogen sulfide producing enzyme and a modulating agent. The modulating agent can be configured to modulate deposition, mode of action, and/or metabolism of a chemotherapeutic agent and/or a reactive oxygen species agent.

In at least one embodiment, a hair care product for treatment and/or prophylaxis of forms of induced alopecia can include a pump spray container containing a topical agent. The topical agent can be configured to up-regulate CYP3A, CSE, CBS, 3MST, or other hydrogen sulfide producing enzymes.

In at least one embodiment, a hair care product for treatment and/or prophylaxis of forms of alopecia can include a pump spray container containing hydrogen sulfide, a hydrogen sulfide donor, or a substrate for a hydrogen sulfide producing enzyme and a modulating agent. The modulating agent can be configured to up-regulate CYP3A, CSE, CBS, 3MST, or other hydrogen sulfide producing enzymes.

In at least one embodiment, a method of treatment and/or prophylaxis of forms of alopecia can include applying an amount of a first composition to at least a portion of a scalp of a person before, during, and/or after chemotherapy and/or radiation therapy. The method can further include diagnosing response with a scalp perfusion test to determine if perfusion is reduced by a predetermined amount. If perfusion is reduced by the predetermined amount, the method may include applying a therapeutic effective amount of the first composition for treatment and/or prophylaxis of forms of alopecia. If perfusion is not reduced by the predetermined amount, the method may further include applying a second composition to at least a portion of the scalp of the person. The method may further include diagnosing response with a scalp perfusion test to determine if perfusion is reduced by a predetermined amount after application of the second composition. If perfusion is reduced by the predetermined amount, the method may further include applying a therapeutic effective amount of the second composition for treatment and/or prophylaxis of forms of alopecia. The first composition may include a concentration of hydrogen sulfide, a hydrogen sulfide donor, or a substrate for a hydrogen sulfide producing enzyme that is less than a concentration of hydrogen sulfide, a hydrogen sulfide donor, or a substrate for a hydrogen sulfide producing enzyme in the second composition.

In at least one embodiment, method of treatment and/or prophylaxis of forms of alopecia can include applying an amount of a first composition to at least a portion of a scalp of a person before, during, and/or after chemotherapy and/or radiation therapy. The method may further include diagnosing response with a scalp perfusion test to determine if perfusion is reduced by a predetermined amount. If perfusion is reduced by the predetermined amount, the method may further include applying a therapeutic effective amount of the first composition for treatment and/or prophylaxis of forms of alopecia. If perfusion is not reduced by the predetermined amount, the method may further include applying a second composition to at least a portion of the scalp of the person. The method may further include diagnosing response with a scalp perfusion test to determine if perfusion is reduced by a predetermined amount after application of the second composition. If perfusion is reduced by the predetermined amount, the method may further include applying a therapeutic effective amount of the second composition for treatment and/or prophylaxis of forms of alopecia. The first composition may include a concentration of hydrogen sulfide, a hydrogen sulfide donor, or a substrate for a hydrogen sulfide producing enzyme and a modulating agent that is less than a concentration of hydrogen sulfide, a hydrogen sulfide donor, or a substrate for a hydrogen sulfide producing enzyme and the modulating agent in the second composition. The modulating agent may be configured to modulate deposition, mode of action, and/or metabolism of a chemotherapeutic agent and/or a reactive oxygen species agent.

In at least one embodiment, a method of treatment and/or prophylaxis of forms of alopecia can include applying an amount of a first composition to at least a portion of a scalp of a person before, during, and/or after chemotherapy and/or radiation therapy. The method may further include diagnosing response with a scalp perfusion test to determine if perfusion is reduced by a predetermined amount. If perfusion is reduced by the predetermined amount, the method may further include applying a therapeutic effective amount of the first composition for treatment and/or prophylaxis of forms of alopecia. If perfusion is not reduced by the predetermined amount, the method may further include applying a second composition to at least a portion of the scalp of the person. The method may further include diagnosing response with a scalp perfusion test to determine if perfusion is reduced by a predetermined amount after application of the second composition. If perfusion is reduced by the predetermined amount, the method may further include applying a therapeutic effective amount of the second composition for treatment and/or prophylaxis of forms of alopecia. The first composition may include a concentration of a topical agent that is less than a concentration of the topical agent in the second composition. The topical agent may be configured to up-regulate CYP3A, CSE, CBS, 3MST, or other hydrogen sulfide producing enzymes.

In at least one embodiment, a method of treatment and/or prophylaxis of forms of alopecia can include applying an amount of a first composition to at least a portion of a scalp of a person before, during, and/or after chemotherapy and/or radiation therapy. The method may further include diagnosing response with a scalp perfusion test to determine if perfusion is reduced by a predetermined amount. If perfusion is reduced by the predetermined amount, the method may further include applying a therapeutic effective amount of the first composition for treatment and/or prophylaxis of forms of alopecia. If perfusion is not reduced by the predetermined amount, the method may further include applying a second composition to at least a portion of the scalp of the person. The method may further include diagnosing response with a scalp perfusion test to determine if perfusion is reduced by a predetermined amount after application of the second composition. If perfusion is reduced by the predetermined amount, the method may further include applying a therapeutic effective amount of the second composition for treatment and/or prophylaxis of forms of alopecia. The first composition may include a concentration of hydrogen sulfide, a hydrogen sulfide donor, or a substrate for a hydrogen sulfide producing enzyme and a modulating agent that is less than a concentration of hydrogen sulfide, a hydrogen sulfide donor, or a substrate for a hydrogen sulfide producing enzyme and the modulating agent in the second composition. The modulating agent may be configured to up-regulate CYP3A, CSE, CBS, 3MST, or other hydrogen sulfide producing enzymes.

In at least one embodiment, a method of treatment and/or prophylaxis of forms of alopecia can include applying a composition to a scalp of a person before, during, and/or after undergoing chemotherapy and/or radiation therapy, the composition comprising a therapeutically effective amount of hydrogen sulfide, a hydrogen sulfide donor, or a substrate for a hydrogen sulfide producing enzyme and an alpha-1 adrenergic receptor (A1AR) agonist. In yet another embodiment, the composition comprising a therapeutically effective amount of hydrogen sulfide, a hydrogen sulfide donor, or a substrate for a hydrogen sulfide producing enzyme and an agent that produces vasoconstriction are used.

In at least one embodiment, a method for treatment and/or prophylaxis of chemotherapy induced alopecia can include a hydrogen sulfide, hydrogen sulfide donor, or a substrate for a hydrogen sulfide producing enzyme that is used in combination with a scalp cooling cap. In yet another embodiment, a device that delivers hydrogen sulfide, hydrogen sulfide donor, or a substrate for a hydrogen sulfide producing enzyme is built into a scalp cooling cap.

The present invention also discloses a system and method for improving the outcome of a hair transplant surgery by optimizing a holding solution to stabilize hair follicle grafts. In yet another embodiment, a system and method for improving viability of a hair follicle graft is described. In yet another embodiment, a system and method for reducing the occurrence of shock hair loss is described. Various formulations are described, as well as, methods to experimentally evaluate the quality of each formulation. In yet another embodiment, a system and method for improving the outcome of a hair transplant surgery by optimizing a holding solution with a hydrogen sulfide donor is described.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In certain embodiments, the topical active agent may be used to reduce and/or inhibit undesired side-effects of chemotherapy and/or radiotherapy, which can include reducing and/or inhibiting hair loss and/or hair shedding due to chemotherapy and/or radiotherapy. In some embodiments, the inventive methods can be used to reduce and/or inhibit perfusion of a chemotherapy agent to an organ or tissue that is noncancerous (for example skin epithelial cells), alopecia, and/or development of CIA. The topical active agent preferably is hydrogen sulfide, a hydrogen sulfide donor, or a substrate for a hydrogen sulfide producing enzyme. Some topical active agents can include sodium sulfide, sodium hydrosulfide, Lawesson's reagent, diallyl trisulfide (DATS), N-acetylcysteine, lmorpholin-4-ium 4 methoxyphenyl (morpholino) phosphinodithioate (GYY4137), Acetyl deacylasadisulfide (ADA), 4-hydroxythiobenzamide (HTB), N-(benzoylthio)benzamide (NSHD-1), JK-1, ATB-346, naproxen-HBTA, 3-mercaptopyruvic acid, sulfanegen, L-cysteine, pyridoxal 5′-phosphate, homocysteine, α-ketoglutarate, D-cysteine, thiosulfate, and pyruvate.

In certain embodiments, the topical active agent may be used to reduce and/or inhibit undesired side-effects of chemotherapy and/or radiotherapy, which can include reducing and/or inhibiting hair loss and/or hair shedding due to chemotherapy and/or radiotherapy. In some embodiments, the inventive methods can be used to reduce and/or inhibit perfusion of a chemotherapy agent to an organ or tissue that is noncancerous (for example skin epithelial cells), alopecia, and/or development of CIA. The topical active agent preferably is hydrogen sulfide, a hydrogen sulfide donor, or a substrate for a hydrogen sulfide producing enzyme. The agent can be delivered in a slow releasing matrix. Examples of slow releasing matrices are, but not limited to, polycaprolactone (PCL) containing JK1nanofibers with H₂S (PCL-JK1), sodium hydrosulfide microparticles (NaHS@MPs), and diallyl trisulfide (DATS) loaded mesoporous silica nanoparticles (MSN-DATS).

Some embodiments of the composition can include other active agents. These may be A1AR agonists. Suitable A1AR agonists for use in the present description include without limitation are phenylephrine, phenylephrine pivalate, amediphrine, synephrine, cirazoline, desvenlafaxine, etilfrine, metaraminol, methoxamine, naphazoline, oxymetazoline, pseudoephrine, m-synephrine, p-synephrine, octopamine, hordenine, tetrahydrozoline, isometheptene, metaraminol, nicergoline, ergonovine, levonordefrin, phendimetrazine, methoxamine, midodrine, clonidine, pergolide, xylometazoline, droxidopa, epinephrine, mephentermine, 4-methoxyamphetamine, benzphetamine, naphazoline, apraclondine, bromocriptine, oxymetazoline, phenylpropanolamine, pseudoephedrine, dipivefrin, noradrenaline, and chloroethylclonidine. In certain embodiments, the A1AR agonist is synephrine, or is phenylephrine, synephrine, oxymetazoline, or methoxamine. In one embodiment, the therapeutic agent is norepinephrine. Another suitable therapeutic agent for use in the inventive methods herein is extract of Bitter orange (Citrus aurantium), which contains synephrine alkaloids and para-octopamine. Other active agents can include vasoconstrictors such as, without limitation, 25I-NBOMe, amphetamines, antihistamines, caffeine, phenylephrine, propylhexedrine, pseudoephedrine.

Some embodiments of the composition can include other active agents. These may be A2AR agonists. Suitable A2AR agonists for use in the present description include without limitation are clonidine, guanfacine, guanabenz, guanoxabenz, guanethidine, xylazine, tizanidine, medetomidine, methyldopa, methylnorepinephrine, fadolmidine, and demedetomidine. Any one or combination of the above-identified agonists may be used in embodiments of the invention.

Some embodiments of the composition can include other active agents. These may be Trace associate amine receptor (TAAR) agonists. Suitable TAAR agonists for use in the present description include without limitation are Citrus aurantium (e.g. bitter orange extract), 2-phenylethylamine, tyramine, p-tyramine, m-tyramine, N-methyltyramine, tryptamine, octopamine, m-octopamine, p-octopamine, ractopamine, dopamine, 5HT, 3-methoxy-tyramine, trimethylamine, dimethylethylamine, N-methylpiperidine, 3-iodothyronamined, N,N-dimethylcyclohexylamine, isoamylamine, cyclohexylamine, serotonin, 3-methoxytyramine, amphetamine-like, amphetamine, methamphetamine, MDMA, cathinone, methcathinone, phenethylamines, N-methylphenethylamine, 2,5-dimethoxy-4-bromo-phenethylamine, 2,5-dimethoxy-4-propyl-phenethylamine, mescaline, (−)-Ephedrine, tryptamines, psilocin, N,N-dimethyltryptamine, ergolines, lysergic acid diethylamide, piperazines, m-chlorophenylpiperazine, aminoindanes, 2-aminoindane, 5-iodo-2-aminoindane, apomorphine, ractopamine, 3-iodothyronamine, clonidine, guanabenz, idazoxan, RO5073012, RO5166017, RO5203648, RO5256390, RO5263397, RO5212773 (EPPTB), etc. Other suitable TAAR agonists can be found at: Mark D. Berry, et al. Pharmacology of Human Trace Amine-associated Receptors: Therapeutic Opportunities and Challenges. Pharmacology & Therapeutics 18 (2017) 161-180, the entire contents of which is incorporated herein by reference in its entirety. Additionally, derivatives of TAAR agonists can be utilized including derivatives of the compounds mentioned above.

Some embodiments of the invention involve co-administration of a topical active agent with a modulating agent, the modulating agent being configured to modulate deposition, mode of action, and/or metabolism of a chemotherapeutic agent and/or a ROS agent. Some embodiments of the invention involve methods to reduce metabolism in rapidly dividing cells. In some embodiments the invention involves administering a topical active agent such as a disclosed herein in combination with administration of a modulating agent configured to up-regulate CYP3A, CSE, CBS, 3MST, or other hydrogen sulfide producing enzymes.

In at least one embodiment, the inventive methods and compositions are for topical application. For example, methods can include reducing and/or inhibiting perfusion or metabolism of a chemotherapy agent to an organ or tissue that is noncancerous, alopecia, and/or CIA by applying a therapeutically effective amount of an embodiment of the composition to the skin (e.g., the scalp or eyebrow) of a person. This can include applying the therapeutic effective amount of an embodiment of the composition to the scalp of a person before, during, and/or after the person undergoes cancer treatment (e.g., receives chemotherapy and/or radiotherapy).

The active agents may be applied in the form of a composition that is a solution, an emulsion, and/or a gel. Some embodiments can include a kit. The kit can include an embodiment of the composition and other ingredient (e.g., shampoo). Some embodiments can include a hair care product. The hair care product can include a pump spray container containing an embodiment of the composition. Some embodiments can include a pharmaceutical preparation. The pharmaceutical preparation can include an embodiment of the composition in a pharmaceutically-acceptable topical delivery vehicle suitable for topically delivering the composition. Some embodiments can include a cap or device that will deliver the embodiment through a scalp-cooling cap.

The present invention may be used in cancer therapy, such as without limitation cancer treatment (e.g., taxmen therapy) for breast cancer and colorectal cancer. However, the present invention can be applicable to most, if not all, types of cancers (e.g., solid organ tumor cancers, sarcomas, carcinoma, lymphomas, etc.) and associated cancer treatments. In at least one embodiment, the present invention can be used as a prophylactic treatment of CIA for patients undergoing cancer treatment with Taxane and/or Anthracycline-based chemotherapy. This can include stage I and/or stage II breast cancer patients.

In some embodiments, the present invention can include use of hydrogen sulfide, a hydrogen sulfide donor, or a substrate for a hydrogen sulfide producing enzyme applied to the scalp or eyebrow. This may be done to reduce and/or reduce cellular metabolism. This may limit the amount of chemotherapeutic agents delivered to the hair follicle. Limiting the amount of chemotherapeutic agents delivered the hair follicle can reduce and/or inhibit CIA.

In some embodiments, the present invention can include use of hydrogen sulfide, a hydrogen sulfide donor, or a substrate for a hydrogen sulfide producing enzyme applied to the cuticle of the fingernail. This may be done to reduce and/or reduce cellular metabolism. This may limit the amount of chemotherapeutic agents delivered to the cuticle of the fingernail. Limiting the amount of chemotherapeutic agents delivered the cuticle of the fingernail can reduce and/or inhibit fingernail loss resulting from chemotherapy.

In some embodiments, the present invention can include use of a topical active agent in combination with a modulating agent topically applied to the scalp. The topical active agent can be hydrogen sulfide, a hydrogen sulfide donor, or a substrate for a hydrogen sulfide producing enzyme or any combination thereof. The modulating agent can be configured to modulate deposition, mode of action, and/or metabolism of a chemotherapeutic agent. A modulating agent can include any one or combination of caffeine, MAD11 monoclonal antibody (MAb), Cyclosporine A, N-acetyl cysteine, alpha-tocopherol, beta-tocopherol, gamma-tocopherol, delta-tocopherol, Calcitriol, CDK2 inhibitor, 2,2′-methylenebis, Antioxidants, Dexamethasone, Praeruptorin C, Rifampin, Carbamezapine, Phenytoin, Phenobarbital, Corticosteroids, St. John's Wart, and Calcitriol-analogs.

In some embodiments, the present invention can include use of hydrogen sulfide, a hydrogen sulfide donor, or a substrate for a hydrogen sulfide producing enzyme applied to the scalp. This may be done to reduce and/or inhibit metabolism. This may limit the amount of ROS agents delivered to the hair follicle. Limiting ROS agents delivered to the hair follicle can reduce and/or inhibit CIA due to radiotherapy.

In some embodiments, the present invention can include use of hydrogen sulfide, a hydrogen sulfide donor, or a substrate for a hydrogen sulfide producing enzyme applied to the scalp. This may be done to reduce and/or inhibit metabolism. This may limit the amount of ROS agents delivered to the hair follicle. Limiting ROS agents delivered to the hair follicle can reduce and/or inhibit CIA when ROS is the mode of action of a chemotherapeutic compound used for chemotherapy treatment.

In some embodiments, the present invention can include use of hydrogen sulfide, a hydrogen sulfide donor, or a substrate for a hydrogen sulfide producing enzyme topically applied to the scalp. With some embodiments, the composition can be applied all at once. Some embodiments involve multiple applications of the composition. In a preferred embodiment, the composition is applied 10-30 minutes prior to chemotherapy and/or radiotherapy and at the end of chemotherapy and/or radiotherapy (e.g., about 2 hours after beginning the chemotherapy and/or radiotherapy). Some embodiments can include applying a therapeutically effective amount of an embodiment of the composition for a prolonged period of time, such as, without limitation, for 48, hours, 24 hours, 12 hours, 6 hours, 5 hours, 4 hours, 3 hours, 2 hours, or one hour, or any amount of time there-between. Some embodiments can include applying the composition repeatedly for the prolonged period of time (e.g., applying the composition every 30 minutes, one hour, 2 hours, 3 hours, 4 hours, etc.). This can include re-applying the composition for up to 48 hours before, during, and/or after undergoing chemotherapy and/or radiotherapy. In some embodiments, the composition can be applied before undergoing chemotherapy and/or radiotherapy treatment and then for a prolonged period of time after undergoing chemotherapy and/or radiotherapy. In some embodiments, the composition can be applied for a prolonged period of time before undergoing chemotherapy and/or radiotherapy treatment and then applied after undergoing chemotherapy and/or radiotherapy. In some embodiments, the composition can be applied for a prolonged period of time before undergoing chemotherapy and/or radiotherapy treatment and then applied for a prolonged period of time after undergoing chemotherapy and/or radiotherapy. Applying the composition for a prolonged period of time may reduce metabolism in local tissue. For example, this may reduce metabolism in rapidly dividing cells. Inducing hypoxia in rapidly dividing cells can reduce the effect of chemotherapeutic agents delivered to the hair follicle. Reducing the effect of chemotherapeutic agents delivered to the hair follicle can reduce the likelihood of developing CIA.

In some embodiments, the present invention can include use of a topical active agent in combination with a modulating agent topically applied to the scalp or eyebrow. The topical active agent can be hydrogen sulfide, a hydrogen sulfide donor, or a substrate for a hydrogen sulfide producing enzyme, or any combination thereof. The modulating agent can be configured to modulate deposition, mode of action, and/or metabolism of a ROS. This may be done to reduce and/or inhibit CIA due to radiotherapy. A modulating agent can include any one or combination of caffeine, MAD11 monoclonal antibody (MAb), Cyclosporine A, N-acetyl cysteine, alpha-tocopherol, beta-tocopherol, gamma-tocopherol, delta-tocopherol, Calcitriol, CDK2 inhibitor, 2,2′-methylenebis, Antioxidants , Dexamethasone, Praeruptorin C, Rifampin, Carbamezapine, Phenytoin, Phenobarbital, Corticosteroids, St. John's Wart, and Calcitriol-analogs.

In some embodiments, the present invention can include use of a first topical agent and a second topical agent topically applied to the scalp, where the second topical agent is configured to up-regulate CYP3A, CSE, CBS, 3MST, or other hydrogen sulfide producing enzymes. This can be done to reduce and/or inhibit CIA due to taxanes (e.g., docetaxel and paclitaxel) that may be used for treatment therapy. A second topical agent can include any one or combination of caffeine, MAD11 monoclonal antibody (MAb), Cyclosporine A, N-acetyl cysteine, alpha-tocopherol, beta-tocopherol, gamma-tocopherol, delta-tocopherol, Calcitriol, CDK2 inhibitor, 2,2′-methylenebis, Antioxidants , Dexamethasone, Praeruptorin C, Rifampin, Carbamezapine, Phenytoin, Phenobarbital, Corticosteroids, St. John's Wart, and Calcitriol-analogs.

In some embodiments, the present invention can include use of a topical active agent and a modulating agent topically applied to the scalp, where the modulating agent is configured to up-regulate CYP3A, CSE, CBS, 3MST, or other hydrogen sulfide producing enzymes. The topical active agent can be hydrogen sulfide, a hydrogen sulfide donor, or a substrate for a hydrogen sulfide producing enzyme, or any combination thereof. This can be done to reduce CIA due to taxanes (e.g., docetaxel and paclitaxel) that may be used for treatment therapy. A modulating agent can include any one or combination of caffeine, MAD11 monoclonal antibody (MAb), Cyclosporine A, N-acetyl cysteine, alpha-tocopherol, beta-tocopherol, gamma-tocopherol, delta-tocopherol, Calcitriol, CDK2 inhibitor, 2,2′-methylenebis, Antioxidants, Dexamethasone, Praeruptorin C, Rifampin, Carbamezapine, Phenytoin, Phenobarbital, Corticosteroids, St. John's Wart, and Calcitriol-analogs.

The topical active agent as used herein can include sodium sulfide, sodium hydrosulfide, Lawesson's reagent, diallyl trisulfide (DATS), N-acetylcysteine, lmorpholin-4-ium 4 methoxyphenyl (morpholino) phosphinodithioate (GYY4137), Acetyl deacylasadisulfide (ADA), 4-hydroxythiobenzamide (HTB), N-(benzoylthio)benzamide (NSHD-1), JK-1, ATB-346, naproxen-HBTA, 3-mercaptopyruvic acid, sulfanegen, L-cysteine, pyridoxal 5′-phosphate, homocysteine, α-ketoglutarate, D-cysteine, thiosulfate, and pyruvate. Embodiments may further relate to methods of preparing the compositions, methods of administering the compositions, methods of treatment or prevention of the effects of hair loss due to chemotherapy and/or radiotherapy by applying or administering therapeutically effective amounts of the compositions, and kits comprising the embodiments of the composition and at least one additional hair care product or container for administering the compositions.

During hair transplantation surgery, follicular grafts are excised from the donor region either as strips, punch biopsy, or with specialized tools that can extract a single follicular unit. Regardless of the method used, the excised grafts are placed in a holding solution, e.g., saline. Strip and punch biopsied hair is later removed from the holding solution and dissected into single follicular units and placed back into the holding solution. The success of current hair transplantation surgeries is limited by the follicular survival rate after implantation. Additionally, transplanted follicular units in anagen phase can experience shock hair loss, which can lead to hairs being pre-maturely converted to telogen phase of the hair cycle after transplantation. To date, there have been no clinically implemented improvements to the holding solutions used in hair transplantation that have adequately addressed shock hair loss. Reducing shock hair loss would vastly improve the results obtained from hair transplantation surgery.

Accordingly, we have developed a number of improvements to hair graft holding solutions to improve the outcomes of hair transplantation surgery. In one embodiment of the present invention a holding solution can be composed of a tissue culture media suitable for culturing cells found in the outer root sheath (ORS) of a hair follicle. In another embodiment of the present invention a holding solution can be composed of a tissue culture media suitable for culturing cells found in the demal papilia (DP) of a hair follicle. In one embodiment of the present invention a holding solution can be composed of an organ culture media suitable for culturing cells found in the outer root sheath (ORS) of a hair follicle. In another embodiment of the present invention a holding solution can be composed of an organ culture media suitable for culturing cells found in the demal papilia (DP) of a hair follicle. In another embodiment of the present invention a holding solution can be composed of an organ culture media suitable for culturing scalp follicular units (FU).

Examples of suitable medias for supporting the viability of hair follicle grafts include but are not limited to: Williams' E Medium, MEM (Minimal Essential Medium Eagle), DMEM (Dulbecco's Modified Eagle's Medium), IMDM (Iscove's Modified Dulbecco's Medium), RPMI-1640 (Roswell Park Memorial Institute Medium), HamF10, HamF12, DMEM/HamF12 (50:50 Dulbecco's Modified Eagle's Medium: HamF12), McCoy's 5A Medium, 199 Medium, Keratinocyte Medium II, hypothermasol, and Ringer's lactate media. Additionally, a modified Eagle Minimum Essential Media (MEM) containing deoxyribonucleosides, ribonucleosides, L-glutamine, penicillin, streptomycin, amphotericin B, and sodium bicarbonate may be used.

In another embodiment of the present invention an excised hair follicle holding solution can be composed of an organ culture media contain a concentration of hydrogen sulfide, a hydrogen sulfide donor, or a substrate for a hydrogen sulfide producing enzyme. Examples of hydrogen sulfide donors include, but are not limited to, sodium sulfide, sodium hydrosulfide, Lawesson's reagent, diallyl trisulfide (DATS), N-acetylcysteine, lmorpholin-4-ium 4 methoxyphenyl (morpholino) phosphinodithioate (GYY4137), Acetyl deacylasadisulfide (ADA), 4-hydroxythiobenzamide (HTB), N-(benzoylthio)benzamide (NSHD-1), JK-1, ATB-346, naproxen-HBTA, 3-mercaptopyruvic acid, sulfanegen, L-cysteine, pyridoxal 5′-phosphate, homocysteine, α-ketoglutarate, D-cysteine, thiosulfate, and pyruvate.

In yet another embodiment of the present invention an excised hair follicle holding solution can be composed of a media containing a chemistry designed to release hydrogen sulfide into the media continuously so that hair follicles can be maintained for long durations up to 12 hours, i.e., slow releasing. Examples of slow releasing chemistries are, but not limited to, polycaprolactone (PCL) containing JK1nanofibers with H₂S (PCL-JK1), sodium hydrosulfide microparticles (NaHS@MPs), and diallyl trisulfide (DATS) loaded mesoporous silica nanoparticles (MSN-DATS).

In another embodiment of the present invention an excised hair follicle holding solution can be composed of an organ culture media containing a chemistry designed to release hydrogen sulfide into the media continuously so that hair follicles can be maintained for long durations up to 12 hours. One such media may contain mesoporous silica nanoparticles (MSN) as the carrier of diallyl trisulfide (DATS). MSN-DATS system can be activated by glutathione to produce controllable hydrogen sulfide release rates.

In another embodiment of the present invention a system of two medias may be arranged such that the first media contains mesoporous silica nanoparticles (MSN) as the carrier of diallyl trisulfide (DATS) and a second media contains glutathione. Such medias can be stored without activating the release of hydrogen sulfide. At a time nearing surgery the medias can be combined to activate the slow release of hydrogen sulfide.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

As used herein, the terms “prevent” or “prevention” and other derivatives of the words, when used in reference to alopecia, e.g., CIA, refer to a reduced likelihood of alopecia in an individual receiving a given treatment relative to that of a similar individual at risk for alopecia but not receiving that treatment. As such, the terms “prevent” and “prevention” encompass a treatment that results in a lesser degree of alopecia than would be otherwise expected for a given individual.

Efficacy for reducing hair loss, inhibiting hair loss, and prevention of alopecia can be established through controlled studies in which a subject is administered a treatment (e.g., a topical treatment) at one site likely to experience or exhibit hair loss or hair shedding but not at another site subjected to the same conditions. Under these circumstances, if the site receiving the topical treatment undergoes less hair loss over time relative to the untreated site, e.g., at least 5% less, at least 10% less, at least 15% less, at least 20% less, at least 25% less, at least 30% less, at least 35% less, at least 40% less, at least 45% less, at least 50% less or beyond, the treatment is effective for reducing hair loss. Determination of efficacy also may involve the measurement or detection of pilomotor stimulation, which can be performed, at its simplest, by observation of the area at the base of the hair shaft to determine whether the smooth muscle of the blood vessels has contracted.

As used herein, the terms “treat,” “treatment,” or “treating” refers to reversing, inhibiting, slowing down or stopping the progression or severity of a disease or condition, e.g., CIA or other form of alopecia. Treatment of alopecia, and particularly CIA, is generally “effective” if hair loss or hair shedding is slowed or stopped, or hair regrows at a faster rate than hair is lost. The methods of showing efficacy for prevention of alopecia discussed above are also applicable for showing efficacy of treatment of alopecia.

As used herein, the term “inhibit” or “inhibiting” in the context of hair loss or hair shedding means to reduce the amount of hair removed or hair that falls out, such as reducing the amount of hair shedding when a cosmetic procedure is applied to the hair.

As used herein the term “perfusion” refers to the passage of fluid through the circulatory system or lymphatic system to an organ or a tissue, usually referring to the delivery of blood to a capillary bed in tissue.

As used herein the term “metabolism” refers to the chemical processes that occur within a living cell that break down or degrade substrates. For chemotherapeutic agents, as used herein the term “metabolism” refers to transport, delivery, and breakdown of chemical compound.

As used herein the term “comprising” or “comprises” is used in reference to compositions, methods, etc. refers to component(s) or method steps that are present in the method or composition, yet allows for the composition, method, etc. to also include unspecified elements.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### Methods of Use

Embodiments of the compositions described herein may be used in methods of treating or preventing various conditions related to hair loss and/or hair shedding, and especially hair loss related to CIA or other forms of alopecia. In one embodiment, therapeutically effective amounts of the compositions described herein are applied topically to the scalp or other body surface containing hair follicles where prevention or treatment of hair loss is desired due to chemotherapy and/or radiotherapy treatment. Embodiments of the composition can be applied before, during, and/or after chemotherapy and/or radiotherapy treatment. Embodiments of composition can be applied multiple times. The same or different composition may be applied when multiple applications are used.

Embodiments of the composition can be liquid solutions. The liquid solution may be applied directly to the scalp and rubbed into the scalp, or applied by spraying on with a delivery device such as a pump sprayer. Embodiments of the composition may further be combined with a shampoo or a conditioner or other hair care product to create a product that has more than one function.

In some embodiments, the compositions described herein are provided as a kit. A “kit” typically defines a package including at least one embodiment of the composition and another item that may be useful in its application, such as a comb, brush or other applicator, or with another hair care composition product, such as a shampoo, hair color/dye, hair oil or conditioner. For example, a kit may be a package containing at least one embodiment of the composition and a spray container or a dropper for administering the composition. In another embodiment, the kit can be a package containing (1) the an embodiment of the composition and (2) one or more of a shampoo, hairspray, conditioner, detangling solution, hair color, henna, or hair oil (such as without limitation coconut oil, jojoba oil, olive oil, baby oil, and black castor oil) and optionally (3) a pump spray container holding an embodiment of the composition or suitable to hold the composition. In another embodiment, the kit may include a container containing an embodiment of the composition and an applicator configured test a region of the scalp of a person for perfusion. Other embodiments of the kit can include a container containing an embodiment of the composition and a disposable device (e.g., a tip) that can be used in conjunction with an applicator, the applicator being configured to test a region of the scalp of a person for perfusion. The applicator in this embodiment may or may not be part of the kit.

### Generating H₂S via Local Flora

It is contemplated for some embodiments to include the use of an agent that can induce local flora to generate H₂S or acceptable variants thereof. For example, an agent such as a probiotic, a genetically modified (GMO) bacteria, or a viral vector can be used to induce local flora in a person in need of treatment so as to cause generation of a H₂S or acceptable variants thereof. Using an agent to induce the generation of H₂S or acceptable variants thereof can be done instead of applying a topical agent containing H₂S or in addition to applying the topical agent containing the H₂S. In some embodiments, methods disclosed herein can be used for identifying a candidate H₂S agent. A probiotic, a GMO bacteria, and/or a viral vector can then be selected based on its ability to cause local flora in a patient to generate the candidate H₂S agent. Use of an agent that can induce local flora to generate H₂S or acceptable variants thereof can be useful for treatments associated with chemotherapy induced alopecia (CIA), for example. However, use of such agents can be used to treat other forms of alopecia as well.

### EXAMPLE

### Assessment of Successful Hair Preservation

Successful hair preservation can be assessed using the Common Terminology Criteria for Adverse Events (CTCAE) version 4.0 scale (see Table 2) at the end of 4 cycles of chemotherapy. In addition, or in the alternative, global photographs can be collected after each chemotherapy cycle and evaluated by an independent physician.

TABLE 2
Skin and subcutaneous tissue disorders

| Adverse | Grade | | | | |
|---|---|---|---|---|---|
| Event | 1 | 2 | 3 | 4 | 5 |
| Alopecia | Hair loss of <50% | Hair loss of >=50% | — | — | — |
| | of normal for that individual | normal for that individual | | | |
| | that is not obvious from a | that is readily apparent | | | |
| | distance but only on close | to others; a wig or hair | | | |
| | inspection; a different hair | piece is necessary if the | | | |
| | style may be required to | patient desires to completely | | | |
| | cover the hair loss but it | camouflage the hair loss; | | | |
| | does not require a wig or | associated with psychosocial | | | |
| | hair piece to camouflage | impact | | | |

Definition: A disorder characterized by a decrease in density of hair compared to normal for a given individual at a given age and body location.

Successful hair prevention trials will be conducted using an embodiment of the composition containing a hydrogen sulfide donor. Successful hair preservation will be assessed using the CTCAE version 4.0 scale, where grade 0 [e.g., no hair loss] or grade 1 [e.g., <50% hair loss not requiring a wig] will be considered successful. This will be assessed at the conclusion of 4 cycles of chemotherapy.

The total number of subjects will be 80. The duration of the study is 8-12 weeks (between 2-3 weeks per treatment cycle). The study will be a double-blinded placebo controlled study. A total of 80 subjects will be enrolled. 30 subjects with breast cancer will be administered the composition and 10 subjects with breast cancer will be administered the placebo vehicle solution. An additional 30 subjects with solid organ cancer will be administered the composition and 10 subjects with solid organ cancer will be administered the placebo vehicle solution.

### Study Phase I: Screening

1. Each potential subject will be screened for the exclusion and inclusion criteria.
Inclusion Criteria
   Stage I or II breast cancer
   Stage I or II solid organ cancer
   Scheduled but not begun, at least 4 cycles of Taxane and/or Anthracycline-based chemotherapy.
   Ages 18-65
Exclusion Criteria
   Subjects with uncontrolled hypertension
   Subjects diagnosed with pattern hair loss or with other hair loss in conjunction with female pattern hair loss
   Folliculitis
   Scalp psoriasis
   Seborrheic dermatitis
   Inflammatory scalp conditions such as lichen planopillaris
   Subjects wearing wigs prior to chemotherapy

2. BP of each subject will be measure and recorded.

### Study Phase II: Chemotherapy Treatment

1. 10-15 minutes prior to each chemotherapy session, 5 mL of the composition will be applied to the entire scalp and it will be massaged into subject's scalp.

2. An additional 5 mL of the composition will be applied to the entire scalp again and massaged into subject's scalp again.

3. The subject will complete a normal chemotherapy treatment session.

4. At the end of each chemotherapy session, 5 mL of the composition will be applied to the entire scalp and massaged into subject's scalp.

5. An additional 5 mL of the composition will be applied to the entire scalp and massaged into subject's scalp again.

6. After completion of each chemotherapy cycle (2-3 weeks), the subject's hair loss will be documented using global photography.

7. A total of 4 cycles of chemotherapy will be documented.

Clinical assessment of efficacy can be performed by assessing global photographs will be collected after each chemotherapy cycle and evaluated by an independent physician. Each test site will be assessed for any potential irritation or sensitisation from the application of the composition, which may include any complaints of adverse events reported by the subjects. The heart rate and blood pressure of each subject using the composition will be closely monitored.

Some embodiments can involve use of a modified CTCAE scale. This can include use of the Dean scale. The following hair loss indicators can be used for the Dean scale
Grade: 0 indicated no hair loss
Grade 1: >0 up to 25% hair loss
Grade 2: >25 up to 50% hair loss
Grade 3: >50 up to 75% hair loss
Grade 4: >75% hair loss

Treatment using the methods disclosed herein can be deemed successful if the maximum Dean score is ≤2 four weeks after the last chemotherapy session. The scoring can be performed by the patient, a physician, or an independent expert based on photography.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

All patents and other publications, including literature references, issued patents, published patent applications, and co-pending patent applications, cited throughout this application are expressly incorporated herein by reference in their entireties.

### REFERENCES

Avram, M. R.; Finney, R.; Rogers, N. (2017) Hair Transplantation Controversies. Dermatologic Surgery Vol. 43, Supplemental 2:S158-S162.

Balsari, A. L.; Morelli, D.; Menard, S.; Veronesi, U. & Colnagho, M. I. (1994) Protection against doxorubicin-induced alopecia in rats by liposome-entrapped monoclonal antibodies. The FASEB Journal Vol. 8, No. 2, (February 1994), pp. 226-230.

Batchelor, D. (2001). Hair and cancer chemotherapy: consequences and nursing care—a literature study. European Journal of Cancer Care Vol. 10, No. 3, (September 2001), pp. 147-163.

Conklin, K. A. (2004) Chemotherapy-associated oxidative stress: impact on chemotherapeutic effectiveness. Integrative Cancer Therapies Vol. 3, No. 4, (December 2004), pp. 294-300.

Coavoy-Sánchez, S. A.; Costa, S. K. P.; Muscará, M. N. (2019) Hydrogen sulfide and dermatological diseases. Br J Pharmacol. 2019 May 3. doi: 10.1111/bph.14699. [Epub ahead of print].

Davis, S. T.; Benson, B. G.; Bramson, H. N.; Chapman, D. E.; Dickerson, S. H.; Dold, K. M.; Eberwein, D. J.; Edelstein, M.; Frye, S. V.; Gampe Jr., R. T.; Griffin, R. J.; Harris, P. A.; Hassel, A. M.; Holmes, W. D.; Hunter, R. N.; Knick, V. B.; Lackey, K.; Lovejoy, B.; Luzzio, M. J.; Murray, D.; Parker, P.; Rocque, W. J.; Shewchuk, L.; Veal, J. M.; Walker, D. H. & Kuyper, L. F. (2001) Prevention of chemotherapy-induced alopecia in rats by CDK inhibitors. Sciences Vol. 291, No. 5501, (January 2001), pp. 134-137.

FDA Press Release:

https://www.fda.gov/newsevents/newsroom/pressannouncements/ucm476216.htm, accessed on Jul. 31, 2017.

Gho, C. G.; Neumann, H. A. (2014) The influence of preservation solution on the viability of grafts in hair transplantation surgery. Plastic and Reconstructive Surgery Vol. 1, No. 9, e90.

Huang L, Wu Q, Li Y, Wang Y, Bi H. PXR-Mediated Upregulation of CYP3A Expression by Herb Compound Praeruptorin C from Peucedanum praeruptorum Dunn. Evid Based Complement Alternat Med. 2013; 2013: 156574.

Hussein, A. M.; Stuart, A. & Peters, W. P. (1995) Protection against chemotherapy-induced alopecia by cyclosporine A in the newborn rat animal model. Dermatology Vol. 190, No. 3, (1995), pp. 192-196.

Jimenez, J. J. & Yunis, A. A. (1996) Vitamin D3 and chemotherapy-induced alopecia. Nutrition Vol. 12, No. 6, (June 1996), pp. 448-449.

McGarvey E L, Baum L D, Pinkerton R C, et al. Psychological sequelae and alopecia among women with cancer. Cancer Pract 9 (6):283-9 (2001 November-December).

Nangia J, Wang T, Osborne C, Niravath P, Otte K, Papish S, Holmes F, Abraham J, Lacouture M, Courtright J, Paxman R, Rude M, Hilsenbeck S, Osborne C K, Rimawi M. Effect of a Scalp Cooling Device on Alopecia in Women Undergoing Chemotherapy for Breast Cancer: The SCALP Randomized Clinical Trial. JAMA. 2017 Feb. 14; 317 (6): 596-605.

Schilli M B, Paus R, Menrad A. Reduction of intrafollicular apoptosis in chemotherapy-induced alopecia by topical calcitriol-analogs. J Invest Dermatol. 1998 October; 111 (4):598-604.

Soref C M, Fahl W E. Int J Cancer. A new strategy to prevent chemotherapy and radiotherapy-induced alopecia using topically applied vasoconstrictor. 2015 Jan. 1; 136 (1): 195-203.

Trueb R M. Chemotherapy-induced alopecia. Semin Cutan Med Surg 28 (1):11-4 (2009 March).

Tsuda, T.; Ohmori, Y.; Muramatsu, H.; Hosaka, Y.; Takigushi, K.; Saitoh, F.; Kato, K.; Nakayama, K.; Nakamura, N.; Nagata, S. & Mochizuki, H. (2001) Inhibitory effect of M50054, a novel inhibitor of apoptosis, on anti-Fas-antibody-induced hepatitis and chemotherapy-induced alopecia. European Journal of Pharmacology Vol. 433, No. 1, (December 2001), pp. 43337-43345.

Wang, J.; Lu, Z. & Au, J. L. S. (2006) Protection against chemotherapy-induced alopecia. Pharmaceutical Research Vol. 23, No. 11, (November 2006), pp. 2505-2514.

de Weger V A, Beijnen J H, Schellens J H. Cellular and clinical pharmacology of the taxanes docetaxel and paclitaxel—a review. Anticancer Drugs. 2014 May; 25 (5):488-94.

What is claimed is:

1. A method of treatment and/or prophylaxis of forms of alopecia, the method comprising:
applying a composition including an agent to reduce and/or inhibit the effects of cancer treatment;
wherein the effects of cancer treatment include hair loss and/or hair shedding due to chemotherapy and/or radiotherapy; and
wherein the agent includes a therapeutically effective amount of hydrogen sulfide, a hydrogen sulfide donor, or a substrate for a hydrogen sulfide producing enzyme molecule.

2. The method of claim 1, further comprising applying the composition via topical administration.

3. The method of claim 1, further comprising at least one of:
reducing and/or inhibiting perfusion or metabolism of a chemotherapy agent;
reducing metabolism in local tissue;
limiting amounts of chemotherapeutic agents and/or reactive oxygen species agents delivered to a hair follicle; and
reducing metabolism in rapidly dividing cells.

4. The method of claim 1, wherein the composition further includes a modulating agent and the method further comprises modulating deposition, mode of action, and/or metabolism of a chemotherapeutic agent and/or a reactive oxygen species agent.

5. The method of claim 4, further comprising reducing metabolism in rapidly dividing cells.

6. The method of claim 1, wherein the composition further includes an alpha-1 adrenergic receptor (A1AR) agonist, alpha-2 adrenergic receptor (A2AR), a trace associate amine receptor (TAAR) agonists, and/or a vasoconstricting agent.

7. The method of claim 1, wherein the composition is within a slow releasing matrix and the method further comprises delivering the composition via the slow releasing matrix.

8. The method of claim 1, further comprising applying the composition via an application kit, wherein:
the application kit includes a spray device, a hair care product, a pump spray container, a scalp cooling cap, and/or a container; and
the composition is: applied separately but in combination with the spray device, the hair care product, the pump spray container, the scalp cooling cap, or the container; and/or built into the spray device, the hair care product, the pump spray container, the scalp cooling cap, or the container.

9. The method of claim 8, wherein the application kit includes at least one applicator for testing a region of the scalp of a person for perfusion and a disposable tip, wherein the method further comprises using the disposable tip with a perfusion testing device.

10. The method of claim 1, further comprising applying the composition to a scalp, eyebrows, and/or fingernail cuticles of a person before, during, and/or after undergoing chemotherapy and/or radiation therapy.

11. The method of claim 10, further comprising at least one of:
reducing metabolism in local tissue;
limiting amounts of chemotherapeutic agents and/or reactive oxygen species agents delivered to a hair follicle;
maintaining a therapeutically effective amount for a prolonged period of time, the prolonged period of time including a time frame with a range from 1 hour to 48 hours;
formulating the composition with a penetration enhancer; and
formulating the composition with a modulating agent configured to modulate deposition, mode of action, and/or metabolism of a chemotherapeutic agent and/or a reactive oxygen species agent.

12. The method of claim 10, wherein:
the form of alopecia is chemotherapy induced alopecia;
the chemotherapy is Taxane and/or Anthracycline-based chemotherapy; and
the chemotherapy is used to treat stage I and/or stage II breast cancer patients.

13. The method of claim 4, wherein the modulating agent is configured to up-regulate CYP3A, CSE, CBS, 3MST, or other hydrogen sulfide producing enzymes.

14. The method of claim 8, wherein the composition further comprises a modulating agent and the method further comprises modulating deposition, mode of action, and/or metabolism of a chemotherapeutic agent and/or a reactive oxygen species agent.

15. The method of claim 14, wherein the modulating agent is configured to up-regulate CYP3A, CSE, CBS, 3MST, or other hydrogen sulfide producing enzymes.

16. A method of treatment and/or prophylaxis of forms of alopecia, the method comprising:
applying an amount of a first composition to at least a portion of a scalp of a person before, during, and/or after chemotherapy and/or radiation therapy;
diagnosing, via a scalp perfusion test, a response to the first composition that indicates whether perfusion is reduced by a predetermined amount, wherein:
when perfusion is reduced by the predetermined amount, applying a therapeutic effective amount of the first composition for treatment and/or prophylaxis of forms of alopecia;
when perfusion is not reduced by the predetermined amount:
applying an amount of a second composition to at least a portion of the scalp;
diagnosing, via a scalp perfusion test, a response to the second composition that indicates whether perfusion is reduced by the predetermined amount, wherein when perfusion is reduced by the predetermined amount, applying a therapeutic effective amount of the second composition for treatment and/or prophylaxis of forms of alopecia;
wherein the first composition includes a first concentration of an agent and the second composition includes a second concentration of the agent, the first concentration being less than the second concentration, and wherein the agent includes hydrogen sulfide, a hydrogen sulfide donor, or a substrate for a hydrogen sulfide producing enzyme molecule.

17. The method of claim 16, wherein the first composition and/or the second composition further includes a modulating agent and the method further comprises modulating the deposition, mode of action, and/or metabolism of a chemotherapeutic agent and/or a reactive oxygen species agent.

18. The method of claim 16, wherein the first composition and/or the second composition further includes a modulating agent and the method further comprises up-regulating CYP3A, CSE, CBS, 3MST, or other hydrogen sulfide producing enzymes.

19. The method of claim 16, wherein the first composition and/or the second composition further includes an alpha-1 adrenergic receptor (A1AR) agonist, alpha-2 adrenergic receptor (A2AR), a trace associate amine receptor (TAAR) agonists, and/or a vasoconstricting agent.

20. A method for improving the outcome of a hair transplant surgery, the method comprising:
optimizing a holding solution with an agent, the agent including a therapeutically effective amount of hydrogen sulfide, a hydrogen sulfide donor, or a substrate for a hydrogen sulfide producing enzyme molecule; and
placing excised hair follicles of a hair follicle graft into the holding solution to: stabilize the hair follicle graft, improve the viability of the hair follicle graft, and/or reduce the occurrence of shock hair loss when the hair follicle graft is transplanted to a target site of a person.

21. The method of claim 20, wherein the holding solution includes a media containing a chemistry designed to controllably release the agent and the method further comprises releasing the agent via the chemistry.

22. A composition, comprising:
an agent configured for treatment and/or prophylaxis of forms of alopecia and/or optimizing a holding solution, the agent comprising a therapeutically effective amount of hydrogen sulfide, a hydrogen sulfide donor, or a substrate for a hydrogen sulfide producing enzyme molecule.

23. The composition of claim 22, wherein:
the composition for treatment and/or prophylaxis of forms of alopecia further includes an alpha-1 adrenergic receptor (A1AR) agonist, alpha-2 adrenergic receptor (A2AR), a trace associate amine receptor (TAAR) agonists, and/or a vasoconstricting agent; and
the composition for optimizing the holding solution further includes an alpha-2 adrenergic receptor (A2AR), a trace associate amine receptor (TAAR) agonists, and/or a vasoconstricting agent.

24. The composition of claim 22, wherein the composition for treatment and/or prophylaxis of forms of alopecia is formulated as a pharmaceutical preparation, a cosmetic, and/or a hair care product.

25. The composition of claim 22, wherein the composition is formulated as a solution, an emulsion, and/or a gel.

26. The composition of claim 22, wherein the composition includes a modulating agent and/or a penetration enhancer.

27. An application kit, comprising:
a spray device, a hair care product, a pump spray container, a scalp cooling cap, and/or a container; and
a composition including an agent configured for treatment and/or prophylaxis of forms of alopecia, the agent comprising a therapeutically effective amount of hydrogen sulfide, a hydrogen sulfide donor, or a substrate for a hydrogen sulfide producing enzyme molecule.

28. The application kit of claim 27, wherein the composition further includes an alpha-1 adrenergic receptor (A1AR) agonist, alpha-2 adrenergic receptor (A2AR), a trace associate amine receptor (TAAR) agonists, and/or a vasoconstricting agent.

29. A method of treatment for chemotherapy induced alopecia (CIA) and/or radiotherapy induced alopecia (RIA), the method comprising:
applying a composition including an agent to reduce and/or inhibit the effects of cancer treatment;
wherein the effects of cancer treatment include hair loss and/or hair shedding; and
wherein the agent includes a therapeutically effective amount of hydrogen sulfide, a hydrogen sulfide donor, or a substrate for a hydrogen sulfide producing enzyme molecule.

30. A method for treatment of alopecia, the method comprising:
administering a therapeutically-effective concentration of a probiotic, a genetic modified bacteria, and/or a viral vector to cause local floral to generate a hydrogen sulfide, a hydrogen sulfide donor, or a substrate for a hydrogen sulfide producing enzyme molecule.
